(19) United States
(12) Patent Application Publication
(10) Pub. No.: US 2018/0036123 A1
(43) Pub. Date: Feb. 8, 2018

(54) DELIVERY DEVICES FOR IMPLANTABLE MEDICAL DEVICES AND METHODS OF MANUFACTURING SAME

(71) Applicant: Medtronic Vascular, Inc., Santa Rosa, CA (US)

(72) Inventor: Declan Costello, Co. Mayo (IE)

(21) Appl. No.: 15/230,525

(22) Filed: Aug. 8, 2016

(51) Int. Cl. A61F 2/24; B22F 3/105; B33Y 50/00; B29C 67/00; B33Y 10/00

(52) U.S. Cl. CPC A61F 2/2427; B29C 67/0088; B33Y 10/00; B33Y 50/00; B22F 3/1055; B29C 67/0059; B29L 2031/7534

(57) ABSTRACT

This disclosure describes, among other things, methods, devices, and systems for non-invasive evaluation of a patient's vascular system and custom design and manufacture of one or more components of a delivery device for delivery and deployment of an implantable medical device, such as a prosthetic heart valve. For example, a delivery sheath can be 3D printed to have varying durometers at one or more areas along its length and/or circumference so that it can more easily bend and flex as it traverses particularly tortuous anatomical features.

### BACKGROUND

The disclosure generally relates to, among other things, devices and methods of manufacturing delivery devices for transcatheter delivery of an implantable medical device, such as a prosthetic heart valve.

A number of implantable medical devices are available for replacement or repair of a conduit, vessel, or organ structure within a patient. Such devices include homografts, xenografts, bioprostheses such as replacement valves, stents, and the like. Many of such devices are implantable with a delivery device via transcatheter procedures, which are procedures where the delivery device, in which or about which the implantable medical device is disposed, is advanced within a vessel, organ structure or other conduit to a desired location where the implantable medical device is deployed. With certain patients, the configuration delivery device may not be suitable to navigate the unique aspects of the patient's anatomy, which can jeopardize a successful outcome.

The disclosure addresses problems and limitations associated with the related art.

### SUMMARY

Various aspects of the disclosure relate to the use of additive manufacturing, otherwise known as three-dimensional (3D) printing technologies, to manufacture a patient-specific delivery sheath of a delivery device configured to deliver and deploy an implantable medical device, such as a prosthetic heart valve. Prior to manufacture of the delivery sheath, a three-dimensional computed tomography (CT) scan of the pertinent vasculature of the patient is analyzed by a clinician to identify tortuous features of the patient's anatomy that the delivery device will need to traverse in order to deliver the implantable medical device. Then, a dataset corresponding to a three-dimensional delivery sheath of the delivery device is prepared and sent to a 3D printer, which forms the three-dimensional delivery sheath of the delivery device using three-dimensional printing technology. Three dimensional printers and related technology provide relatively inexpensive manufacture of a delivery sheath having a patient-specific variation of durometers at one or more various locations along a length and/or circumference of the delivery sheath based on the patient's particular anatomical features so that the delivery sheath can perform specific bend and flex actions as it moves through a patient's vasculature. Once the delivery sheath is printed, an optional capsule is coupled or otherwise attached to the end of the delivery sheath via a thread, similar coupling or could alternatively be an integrally formed part of the delivery sheath. The delivery sheath and optional capsule form a delivery sheath assembly. The delivery sheath assembly is loaded over an inner shaft assembly of the remainder of a premanufactured delivery device for insertion within the patient along with additional components of the delivery device. The inner shaft assembly is generally flexible and takes the shape formed by the delivery sheath. Alternatively, the delivery sheath can be a separate sheath positioned over both an outer sheath that is attached to a capsule and also the inner shaft assembly. In essence, the delivery sheath is the outermost sheath of the delivery device and components of the delivery device positioned within the delivery sheath are sufficiently flexible to take the shape of the custom manufactured delivery sheath. Patient-specific delivery sheaths disclosed herein are advantageous for achieving proper placement of the implantable medical device in the proper location, which can have many advantages including minimizing the risk of heart block, vascular trauma and/or paravalvular leakage in prosthetic heart valve applications.

### BRIEF DESCRIPTION OF THE DRAWINGS

FIG. 1A is a schematic side view of an embodiment of a prosthetic heart valve in a natural, expanded arrangement.

FIG. 1B is a schematic side view of the prosthetic heart valve of FIG. 1A in a compressed or collapsed arrangement.

FIG. 2A is a partially-exploded, perspective view of an embodiment of a delivery device configured to deliver an implantable medical device, such as the prosthetic heart valve of FIGS. 1A-1B.

FIG. 2B is an assembled, top view of the delivery device of FIG. 2A.

FIG. 3 is a partially-exploded, perspective view of an alternate embodiment of a delivery device configured to deliver an implantable medical device, such as the prosthetic heart valve of FIGS. 1A-1B.

FIG. 4 is a partial, cross-sectional, schematic view of a delivery sheath for use with the delivery device of FIG. 2A or 3.

FIG. 5 is a partial, cross-sectional, schematic view of an alternate delivery sheath for use with the delivery device of FIG. 2A or 3.

FIG. 6 is a partial, schematic view of an alternate delivery sheath for use with the delivery device of FIG. 2A or 3.

FIG. 7 is a partial, cross-sectional, schematic view of an alternate delivery sheath for use with the delivery device of FIG. 2A or 3.

FIG. 8 is a schematic drawing illustrating the use of the example delivery device of FIG. 2A-2B or 3 for transcatheter delivery of an implantable medical device, such as the prosthetic heart valve of FIGS. 1A-1B (not visible).

FIGS. 9A-9B are example CT scans of a patient's vasculature.

FIG. 10 is flow chart illustrating one embodiment of a method of designing and manufacturing a delivery sheath for a delivery device, such as the delivery devices of FIG. 2A-2B or 3.

### DETAILED DESCRIPTION

Implantable medical devices disclosed herein may be expandable from a collapsed or compressed configuration to an expanded configuration and may interact with the interior wall of a vessel, organ structure, or other bioprosthetic or natural conduit, or the like via interference fit when expanded. Examples of expandable implantable medical devices include prosthetic heart valves, stents, grafts and the like.

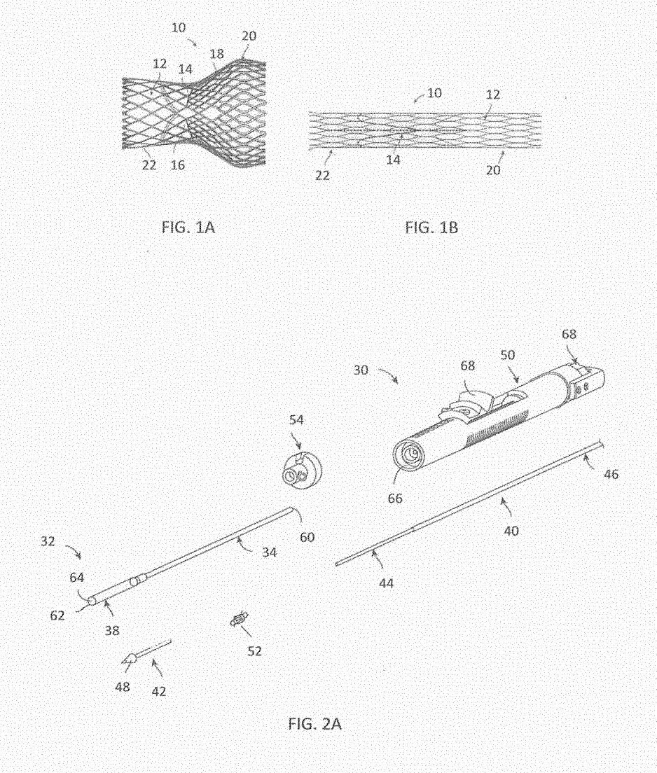

By way of example, one non-limiting example of a prosthetic heart valve 10 useful with devices and methods of the present disclosure is illustrated in FIGS. 1A-1B. As a point of reference, the prosthetic heart valve 10 is shown in a natural or expanded arrangement in the view of FIG. 1A. FIG. 1B illustrates the prosthetic heart valve 10 in a compressed arrangement (e.g., when compressively retained within a delivery sheath or the like). The prosthetic heart valve 10 includes a stent or stent frame 12 and a valve structure 14. The stent frame 12 can assume a variety of forms and is generally constructed so as to be self- or otherwise-expandable from the compressed arrangement (FIG. 1B) to the natural, expanded arrangement (FIG. 1A). The valve structure 14 is assembled to the stent frame 12 and forms or provides two or more (typically three) leaflets 16. The valve structure 14 can also take a variety of forms and can be assembled to the stent frame 12 in various manners, such as by sewing the valve structure 14 to one or more of the wire segments 18 defined by the stent frame 12.

One acceptable construction of the prosthetic heart valve 10 depicted in FIGS. 1A and 1B can be used for repairing a native heart valve. Of course, other shapes and sizes are envisioned to adapt to the specific anatomy of the valve to be repaired (e.g., prosthetic heart valves in accordance with the present disclosure can be shaped and/or sized for replacing a native mitral, aortic, or tricuspid valve). In the depicted embodiment, the valve structure 14 extends less than the entire length of the stent frame 12. An outflow region 20 of the prosthetic heart valve 10 is generally free of the valve structure 14 material, with the valve structure 14 extending along an inflow region 22 of the prosthetic heart valve 10. As a point of reference, “inflow” and “outflow” terminology is in reference to an arrangement of the prosthetic heart valve 10 upon final implantation relative to the native valve being repaired. A wide variety of constructions are also acceptable and within the scope of the present disclosure. For example, in other embodiments, the valve structure 14 can extend along an entirety, or a near entirety, of a length of the stent frame 12.

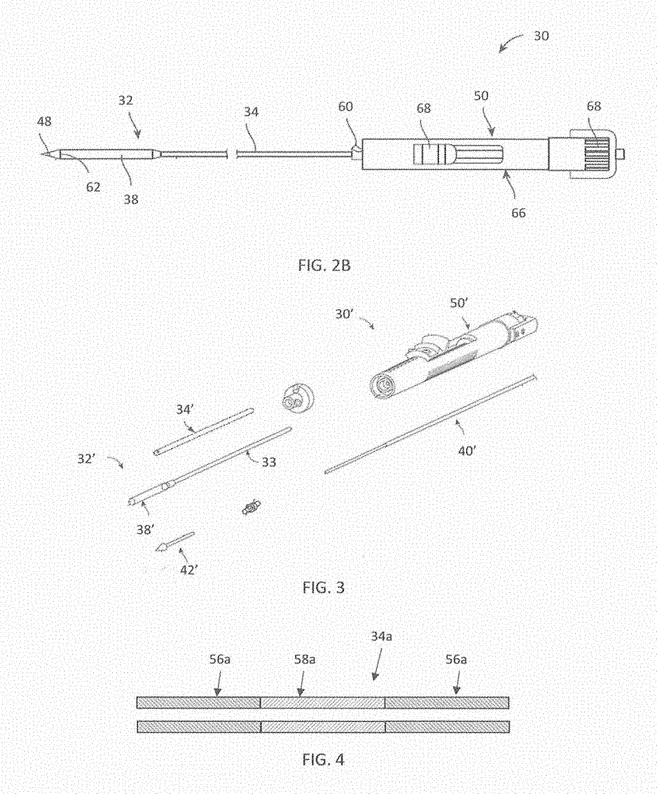

A delivery device 30 for percutaneously delivering the prosthetic heart valve 10 of FIGS. 1A-1B or other implantable medical device is shown in simplified form in FIGS. 2A and 2B. In this illustrative embodiment, the delivery device 30 includes a delivery sheath assembly 32, an inner shaft assembly 40 and a handle assembly 50. Details on the various components are provided below. In general terms, however, the delivery device 30 combines with a prosthetic heart valve or other implantable medical device (not shown) to form a system for performing a therapeutic procedure (e.g., on a defective heart valve of a patient). The delivery device 30 provides a loaded or delivery state in which the prosthetic heart valve is loaded over a support shaft 42 of the inner shaft assembly 40 and is compressively retained within a capsule 38 of the delivery sheath assembly 32 include or provide a valve retainer 52 configured to selectively receive a corresponding feature (e.g., posts) provided with the prosthetic heart valve stent frame. The delivery sheath assembly 32 can be manipulated to withdraw the capsule 38 proximally from over the prosthetic heart valve via operation of the handle assembly 50, permitting the prosthetic heart valve to self-expand and partially release from the support shaft 42. When the capsule 38 is retracted proximally beyond the valve retainer 52, the prosthetic heart valve can completely release or deploy from the delivery device 30. The delivery device 30 can optionally include other components that assist, facilitate or control complete deployment of the implantable medical device. For example, the delivery device 30 can optionally include additional components or features, such as a flush port assembly 54, a recapture sheath (not shown), ability to steer or articulate etc.

Various features of the components 32, 40 and 50 reflected in FIGS. 2A and 2B and as described below can be modified or replaced with differing structures and/or mechanisms. Thus, the present disclosure is in no way limited to the delivery sheath assembly 32, the inner shaft assembly 40, or the handle assembly 50 as shown and described below. Any construction that generally facilitates loading of an implantable medical device for transcatheter delivery via a patient's vasculature is acceptable.

In some embodiments, the delivery sheath assembly 32 defines proximal and distal ends 60, 62, and includes the capsule 38 and a delivery sheath 34. The delivery sheath assembly 32 can be akin to a sheath, defining a lumen 64 (referenced generally) that extends from the distal end 62 through the capsule 38 and at least a portion of the delivery sheath 34. The lumen 64 can be open at the proximal end 60 (e.g., the delivery sheath 34 can be a tube). The capsule 38 extends distally from the delivery sheath 34, and in some embodiments has a more stiffened construction (as compared to a stiffness of the delivery sheath 34) that exhibits sufficient radial or circumferential rigidity to overtly resist the expected expansive forces of the prosthetic heart valve (not shown) when compressed within the capsule 38. For example, the delivery sheath 34 can be a polymer tube, whereas the capsule 38 includes a laser-cut metal tube that is optionally embedded within a polymer covering. Alternatively, the capsule 38 and the delivery sheath 34 can have a more uniform or even homogenous construction (e.g., a continuous polymer tube). Regardless, the capsule 38 is constructed to compressively retain the prosthetic heart valve at a predetermined diameter when loaded within the capsule 38, and the delivery sheath 34 serves to connect the capsule 38 with the handle assembly 50. The delivery sheath 34 (as well as the capsule 38) is constructed to be sufficiently flexible for passage through a patient's vasculature, yet exhibits sufficient longitudinal rigidity to effectuate desired axial movement of the capsule 38. Proximal retraction of the delivery sheath 34 is directly transferred to the capsule 38 and causes a corresponding proximal retraction of the capsule 38. In certain embodiments, the delivery sheath 34 is further configured to transmit a rotational force or movement onto the capsule 38. In this embodiment, the delivery sheath 34 is custom printed to be particularly capable of navigating a patient's specific vasculature, as will be further discussed below.

In some embodiments, the inner shaft assembly 40 includes a proximal shaft or tube 46, an intermediate shaft or tube 44 and the support shaft 42 that terminates at a tip 48. The support shaft 42 is sized to be slidably received within the lumen 64 of the delivery sheath assembly 32 and exhibits sufficient structural integrity to support a loaded, compressed implantable medical device (not shown). The tip 48 forms or defines a nose cone having a distally tapering outer surface adapted to promote atraumatic contact with bodily tissue. The tip 48 can be fixed or slidable relative to the support shaft 42. The intermediate tube 44 is optionally formed of a flexible polymer material (e.g., PEEK) with or without a metal braid, and is sized to be slidably received within the delivery sheath assembly 32. The intermediate tube 44 in some embodiments is a flexible polymer tubing (e.g., PEEK) having a diameter slightly less than that of the proximal tube 46. The proximal tube 46 can have a more rigid construction, configured for robust assembly with the handle assembly 50, such as a metal hypotube. Other constructions are also envisioned. For example, in other embodiments, the intermediate and proximal tubes 44, 46 are integrally formed as a single, homogenous tube or shaft. Regardless, the inner shaft assembly 40 forms or defines at least one lumen (not shown) sized, for example, to slidably receive a guide wire (not shown). The inner shaft assembly 40 can also define a continuous lumen (not shown) sized to slidably receive an auxiliary component such as a guide wire (not shown).

The handle assembly 50 generally includes a housing 66 and one or more actuator mechanisms 68 (referenced generally). The housing 66 maintains the actuator mechanism(s) 68, with the handle assembly 50 configured to facilitate sliding movement of the capsule 38 relative to other components (e.g., the inner shaft assembly 40 and its support shaft 42). The housing 66 can have any shape or size appropriate for convenient handling by a user.

An alternate delivery device 30′ is schematically illustrated in FIG. 3. The delivery device 30′ is configured and operates similar to the delivery device 30 of FIGS. 2A-2B with the exception that the delivery device 30′ includes a delivery sheath assembly 32′ having both an outer sheath 33 and a delivery sheath 34′. As shown in FIG. 3, the delivery device 30′ includes an inner shaft assembly 40′ having an inner shaft 42′ over which an implantable medical device (not shown) can be positioned. The outer sheath 33 includes a capsule 38′, that is configured to compressively retain the implantable medical device (not shown) and the movement of which is controlled by a handle assembly 50′. In this embodiment, the delivery sheath 34′ is custom printed to be particularly capable of navigating a patient's specific vasculature, as will be further discussed below. Once printed, the delivery sheath 34′ is then secured over the outer sheath 33 and capsule 38′, which are positioned over the inner shaft assembly 40′.

A mass produced, “one size fits all” delivery device can accommodate many patients, however, some patients can benefit from a custom device better suited for their particular anatomical features. For example, older patients having scoliosis can have a particularly tortuous anatomy. Therefore, the disclosed delivery devices and methods provide a custom produced delivery sheath designed specifically to navigate through the patent's individual anatomical features. The delivery sheath is designed and manufactured to have a variable durometer or stiffness along its length and/or circumference, which is configured to be aligned with the patient's anatomical features so that the delivery sheath can bend and flex at certain points during delivery of the implantable medical device. Therefore, the delivery devices disclosed herein are better suited to navigate through the patent's particular vasculature, thus generally resulting in more successful outcomes.

FIG. 4 schematically illustrates one example of how a delivery sheath 34a, similar to the delivery sheaths 34, 34′, can be designed to have varying stiffness at one or more areas of the delivery sheath 34a. In this embodiment, the delivery sheath 34a is manufactured to have one or more areas made of a first material 56a having an increased stiffness relative to an adjacent area or section 58a made of a second material. It is to be understood that the more flexible areas made of the second material 58a can be positioned along as many or as few areas along the length of the delivery sheath 34a, as desired. Moreover, the area of lesser stiffness 58a can extend along the entirely of the circumference of the area 58a, or, alternatively, can be irregular or extend along a portion or less than the entirety of the circumference of the delivery sheath 34a. The disclosure is not intended to be limited to any specific configuration in which the first and second materials 56a, 58b can be arranged along the length and/or circumference of the delivery sheath 34a.

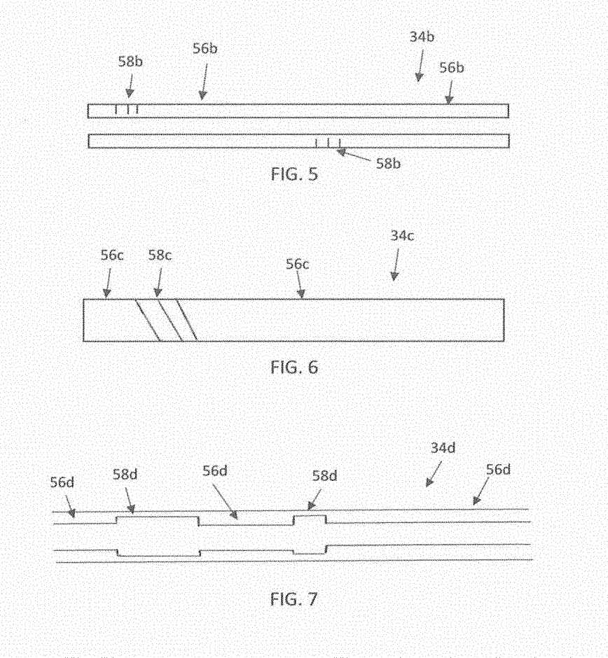

FIG. 5 schematically illustrates another example of how a delivery sheath 34b, similar to delivery sheaths 34, 34′, can be designed to have varying stiffness at one or more areas of the delivery sheath 34b. In this embodiment, the delivery sheath 34b is manufactured to have one or more areas 56b having an increased stiffness relative to an adjacent area or section 58b having a lesser stiffness resulting from one or more cuts (generally referenced by 58b) extending through a partial thickness of the delivery sheath 34b. It will be understood that the “cuts”, in this embodiment, are formed by printing as described herein. The number and location of cuts can vary, as desired, to create areas 58b having a lesser stiffness or greater flexibility as compared to adjacent areas 56b that do not include cuts. As shown, the area of lesser stiffness 58b can extend along the entirety of the circumference of the area 58b, or, alternatively, can be irregular or extend along a portion of the circumference of the delivery sheath 34b. The disclosure is not intended to be limited to any specific configuration in which the stiffness can vary along the length and/or circumference of the delivery sheath 34b.

FIG. 6 schematically illustrates yet another example of how a delivery sheath 34c, similar to delivery sheaths 34, 34′, can be designed to have varying stiffness at one or more areas of the delivery sheath 34c. In this embodiment, the delivery sheath 34c is manufactured to have areas 56c having an increased stiffness relative to an adjacent area or section 58c. The variance in stiffness is a result of one or more spiral cuts (generally referenced by 58c) printed into a partial thickness of the delivery sheath 34c. It will be understood that the “cuts”, in this embodiment, are formed by printing as described herein. The number, length and location of cuts can vary, as desired, to create areas 58c having a lesser stiffness or greater flexibility as compared to adjacent areas 56c that do not include cuts. As shown, the area of lesser stiffness 58a resulting from the cuts can extend along the entirely of the circumference of the area 58c, or, alternatively, can be irregular or extend along a portion of the circumference of the delivery sheath 34c. The disclosure is not intended to be limited to any specific configuration in which the stiffness can vary along the length and/or circumference of the delivery sheath 34c.

FIG. 7 schematically illustrates one example of how a delivery sheath 34d, similar to delivery sheaths 34, 34′, can be custom designed to have varying stiffness at one or more areas of the delivery sheath 34d. In this embodiment, the delivery sheath 34d is designed and manufactured to have areas made of a single material, the areas including an area of increased stiffness 56d relative to an adjacent area or section 58d. In this embodiment, the variance in stiffness between areas 56d, 58d is a result of the delivery sheath 34d having a variance in a wall thickness. It is to be understood that the areas having a lesser stiffness 58d can be positioned along as many or as few areas along the length of the delivery sheath 34d, as desired. Moreover, the area of lesser stiffness 58d can be irregular or extend along the entirely of the circumference of the delivery sheath 34d, or, alternatively, can extend along a portion of the circumference of the delivery sheath 34d. The disclosure is not intended to be limited to any specific configuration in which the stiffness can vary along the length and/or circumference of the delivery sheath 34d.

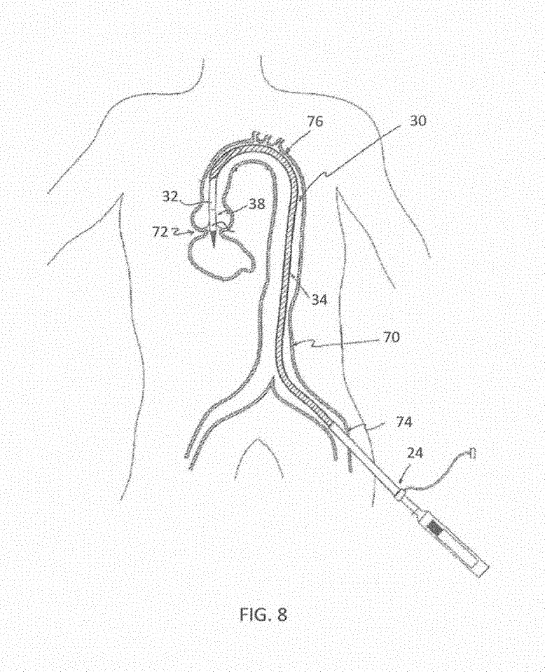

A prosthetic heart valve, such as that depicted in FIGS. 1A-B, or other implantable medical device, may be implanted via a transcatheter procedure. Part of one such procedure is schematically reflected in FIG. 8 in which the delivery device 30 is employed to repair a defective aortic valve 72. As shown, the delivery device 30 (in the loaded state having a loaded prosthetic valve, which is not visible) is introduced into the patient's vasculature 70 (referenced generally) via an introducer device 24. The introducer device 24 provides a port or access to a femoral artery 74. From the femoral artery 74, the delivery device 30 (that compressively retains the implantable medical device) is advanced via a retrograde approach through an aortic arch 76 (e.g., via iliac arteries). FIG. 8 depicts the delivery sheath assembly 32 having the delivery sheath 34 extending along a substantial length of delivery sheath assembly 32, with a distal end of the delivery sheath 34 being fairly proximate to the capsule 38 retaining the prosthetic heart valve. Deployment of the prosthetic heart valve from the delivery device 30 can be accomplished via proximal retraction of the delivery sheath 34, and, in this particular embodiment, the capsule 38.

In various embodiments described herein, one or more characteristics or dimensions of a vessel, organ structure or other bioprosthetic or natural conduit is assessed, measured or determined. As used hereinafter, “vasculature” will be used to collectively refer to a natural conduit, vessel, or organ structure into which an expandable, implantable medical device may be implanted via transcatheter procedure. In various embodiments described herein, one or more maximum and minimum characteristics or dimensions such as diameters, perimeters, lengths, areas, including cross-sectional area or surface area, etc., of a conduit are determined by imaging a portion of the conduit into which the device is to be implanted at appropriate points in the cardiac cycle (or other appropriate cycle, such as the respiratory cycle, etc.). As used herein, “dimensional characteristic” or “anatomical features” will be used to refer collectively to perimeter, diameter (including perimeter derived diameter, area derived diameter, average diameter, major diameter, minor diameter, etc.), area (such as cross-sectional area, surface area, etc.), length, aspect ratio, shape and the like. For example, the dimensional characteristics of the patient's vasculature within the portion of interest may be evaluated and compared to various delivery sheath configurations.

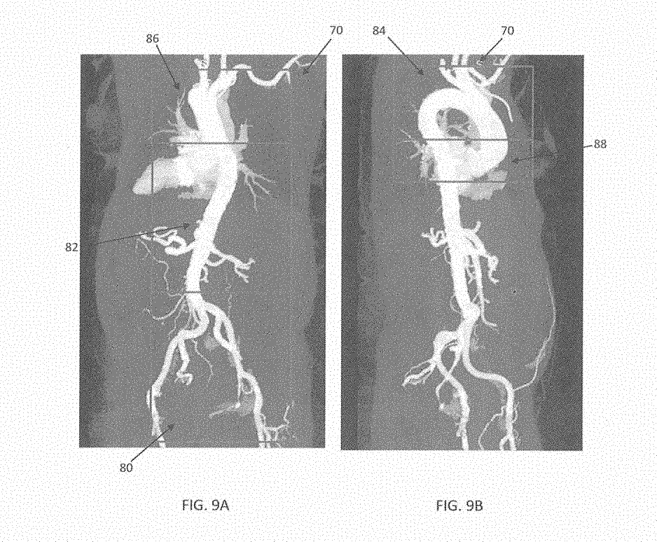

To design and manufacture one of the many delivery sheath embodiments disclosed herein, a three-dimensional computed tomography scan (CT scan) of the patient's vasculature 70 or natural conduit through which the delivery device must travel is obtained. As an example, FIGS. 9A-9B illustrate, in two dimensions, an example CT scan of a patient's vasculature pertinent for transcatheter prosthetic heart valve implantation procedure. The CT scans of FIGS. 9A-9B are annotated to generally identify five areas of interest including the patient's femoral tortuosity 80, length of the descending aorta 82, length, angulation and curvature of the aortic arch 84, length of the ascending aorta 86 and angulation of the aortic annulus 88. From the CT scan, one or more three-dimensional tortuosities or other anatomical features can be identified in the delivery path. A centerline tortuosity can be used to identify the delivery path and the areas of greatest curvature can correlate to the desired areas of greatest flexibility on the delivery sheath. In certain embodiments, the delivery sheath will be designed to be more flexible on an inner surface (with respect to the patient) of the delivery sheath to traverse the aortic arch. After identifying anatomical features that would prove challenging for delivery of the implantable medical device, a patient-specific delivery sheath can be designed including one or more of variances in stiffness (durometer) along the length and/or circumference of the delivery sheath. In this way, the patient-specific delivery device and implantable medical device delivered therewith, can more easily navigate the patient's particular vasculature.

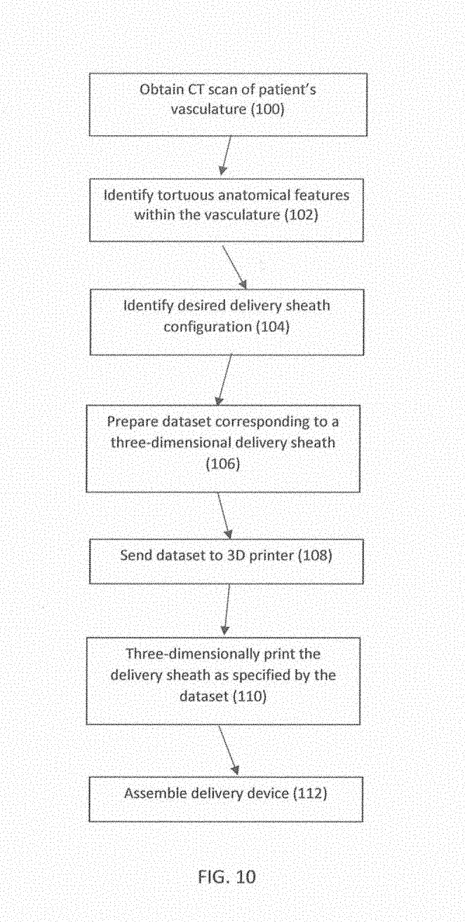

FIG. 10 is a flow chart showing an embodiment of an example method of forming one of the delivery sheaths disclosed herein. The methods as described with respect to FIG. 10 include methods for making a delivery sheath using “three-dimensional printing” (3D printing) or “additive manufacturing” or “rapid prototyping”. The term “three-dimensional printing” or “additive manufacturing” or “rapid prototyping” refers to a process of making a three-dimensional solid object of virtually any shape from a dataset. 3D printing is achieved using an additive process, where successive layers of material are laid down in different shapes. Any type of 3D printing machine that can print the materials described herein may be used.

One initial step in the method is to obtain a CT scan of at least a portion of the pertinent vasculature of the patient 100. As also discussed above with respect to FIGS. 9A-9B, the CT scan is reviewed by a clinician to identify tortious anatomical features of the patent's anatomy within the vasculature that will have been traversed by the delivery device 102. Then, a delivery sheath design is prepared 104. The delivery sheath is designed to have a varying durometer about at least one area of the delivery sheath's circumference and/or along the length of the delivery sheath so that the delivery sheath can appropriately bend and flex as it moves through the patient's vasculature to deliver the implantable medical device. A dataset is then prepared corresponding to the three-dimensional delivery sheath 106.

For example, and not by way of limitation, the dataset may be a 3D printable file such as an STL file. STL (STereoLithography) is a file format native to the stereolithography CAD software created by 3D Systems. STL is also known as Standard Tessellation Language. This file format is supported by many software packages for use in 3D printing. The dataset is sent to a 3D printer that subsequently forms or “prints” the delivery sheath as specified by the dataset 108. In step 110, the 3D printing machine lays down successive layers of a powder or other form of the desired materials to build the delivery sheath from a series of cross sections. The materials used to form the delivery sheath include the material desired for the finished delivery sheath (also referred to as a “structural material”).

Examples of structural materials which may be 3D printed to form the delivery sheath include any biocompatible material, for example, stainless steel (such as “SS316L”), cobalt-chromium alloys, nickel titanium alloys such as Nitinol, magnesium and magnesium alloys, or combinations thereof. The term “cobalt-chromium” alloys as used herein includes alloys with cobalt and chromium. Generally, materials such as, but not limited to, cobalt-nickel-chromium alloys (“MP35N”, “MP20N”, and “MP35NLT”) and chromium-nickel-tungsten-cobalt alloys (“L605”) and cobalt-chromium-nickel-molybdenum alloys (“ELGILOY”) are the types of materials included in the term “cobalt-chromium alloys” as used herein. Polymers may also be used as structural materials to form the delivery sheath. Polymers which may be used to form the delivery sheath include, but are not limited to, polylactide, poylglycolide, polysaccharides, proteins, polyesters, polyhydroxyalkanoates, polyalkelene esters, polyamides, polycaprolactone, polyvinyl esters, polyamide esters, polyvinyl alcohols, modified derivatives of caprolactonepolymers, polytrimethylene carbonate, polyacrylates, polyethylene glycol, hydrogels, photo-curable hydrogels, terminal diols, and combinations thereof.

Once the custom delivery sheath is formed via the 3D printer, the delivery sheath can be assembled to the delivery device, over the inner shaft assembly. As also discussed above, the implantable medical device is positioned on the support shaft in a compressed arrangement so that the delivery sheath can be positioned over the implantable medical device and the inner shaft assembly. In some embodiments, the delivery sheath will compressively retain the implantable medical device onto the support shaft and in alternate embodiments, the capsule will be secured to the distal end of the delivery sheath and will compressively retain the implantable medical device over the support shaft. The capsule can be secured to the delivery sheath via a thread (not shown) or similar coupling or could alternatively, in some embodiments, be an integral part of the delivery sheath. In even further alternate embodiments, as discussed above with respect to FIG. 3, the delivery sheath can be positioned over an outer sheath, wherein the outer sheath is connected to the capsule and positioned over the inner shaft. The delivery device can be sterilized as per normal manufacturing processes or alternatively the delivery device can be sterilized through in hospital methods (autoclave etc.). In other words, after printing the delivery sheath in the hospital, the delivery device can be sterilized and assembled in the hospital, as opposed to a manufacturing facility.

Although the present disclosure has been described with reference to preferred embodiments, workers skilled in the art will recognize that changes can be made in form and detail without departing from the spirit and scope of the present disclosure.

What is claimed is:

1. A method of manufacturing a delivery device for delivering an implantable medical device in a patient via the patient's vasculature, the method comprising the steps of:
providing a three-dimensional computed tomography scan of at least a portion of the patient's vasculature;
reviewing the scan to evaluate anatomical features of the patient's vasculature;
preparing a dataset corresponding to a three-dimensional delivery sheath of the delivery device corresponding to the anatomical features; and
forming the three-dimensional delivery sheath of the delivery device using three-dimensional printing.

2. The method of claim 1, wherein the delivery device further includes an inner shaft assembly; the method further including the step of securing the implantable medical device over the inner shaft assembly.

3. The method of claim 2, further comprising the step of assembling a capsule to a distal end of the delivery sheath.

4. The method of claim 3, wherein the method further comprising assembling the delivery sheath over the inner shaft assembly such that the capsule covers the implantable medical device.

5. The method of claim 2, the method further comprising assembling the delivery sheath to be positioned over both an outer sheath and the inner shaft assembly.

6. The method of claim 1, wherein the delivery sheath is formed to have a variation of durometer along its length.

7. The method of claim 1, wherein the delivery sheath is formed to have a variation of durometer about its circumference.

8. The method of claim 1, wherein the delivery sheath is formed to have a variation of durometer about its circumference and along its length.

9. The method of claim 1, wherein the delivery sheath is formed to have a variation in thickness.

10. The method of claim 1, wherein the delivery sheath includes sections composed of different materials.

11. The method of claim 10, wherein at least two sections composed of different materials have differing durometers.

12. The method of claim 11, wherein a capsule is secured to the outer sheath, the capsule configured to receive and retain the implantable medical device.

13. The method of claim 1, wherein the implantable medical device is a prosthetic heart valve for delivery to a native valve of the patient's heart.

14. The method of claim 1, wherein a plurality of cuts are formed within the delivery sheath to provide a variance in flexibility along a length of the delivery sheath.

15. The method of claim 1, wherein a spiral cut is formed along at least part of a length of the delivery sheath to provide a variance in flexibility along a length of the delivery sheath.
